(19) United States
(12) Patent Application Publication
(10) Pub. No.: US 2022/0160876 A1
(43) Pub. Date: May 26, 2022

(54) MACROMOLECULAR SYSTEM FOR DELIVERY OF EXOGENOUS ACTIVE SUBSTANCES WITH IMMUNE TRAP TO AVOID NEUTRALIZATION THEREOF

(71) Applicants: Igor Verba, Mariupol (UA); Timur Normukhamedov, Friusa (DO)

(72) Inventors: Igor Verba, Mariupol (UA); Timur Normukhamedov, Friusa (DO)

(21) Appl. No.: 17/104,170

(22) Filed: Nov. 25, 2020

(51) Int. Cl. A61K 47/24; A61K 9/10; A61K 47/34; A61K 31/015; A61K 31/355

(52) U.S. Cl. CPC A61K 47/24; A61K 9/10; A61K 31/355; A61K 31/015; A61K 47/34

(57) ABSTRACT

A method for making a macromolecular system for delivery of active substances with an immune trap to avoid neutralization of the substances being administered is disclosed. The method includes the steps of solubilizing the selected active substance in a solubilizer, making a suspension in the form of a nano-dispersed composition of the active substance, and introducing phosphotidylcholine into said composition and mixing with water or an aqueous solution in pre-determined ratios.

### TECHNICAL FIELD

Embodiments disclosed herein relate generally to the chemical and pharmaceutical industry and medicine, and more specifically to methods for making medicinal and veterinary products.

### BACKGROUND

The essence of this invention is that the method for making acidic insulin products having increased stability includes homogenization of the insulin component in surfactants selected from the group consisting of Span®, Tween®, Myrj®, Triton®, Cremophor®, and selected from the group consisting of Poloxamers, Pluronics and Tektronix.

The pharmaceutical composition further includes a preservative selected from the group consisting of phenol, cresol, chlorocresol, benzyl alcohol and paraben; an isotonic agent selected from the group consisting of mannitol, sorbitol, lactose, dextrose, trehalose, sodium chloride and glycerol; buffers such as, for example, TRIS, phosphate, citrate, acetate, glycylglycine, or other substances such as acids, alkalis, and salts.

The resulting products are stable and suitable for use for at least 1 year (they remain undegraded for a long time and can be used independently for making insulin products, and as a source concentrate in complex pharmaceutical formulations).

The disadvantages of the known methods include a relatively high cost of making the products, due to the large number of additional substances used in this method. It should also be noted that Tween-20® and Tween-80® are generally taken as surfactants, which are known to cause allergic responses.

The objective of the subject invention is to provide a macromolecular system for delivery of exogenous active substances that can be used in the manufacture of products with various desired properties. The disadvantage of such existing systems is the neutralization of the substances being introduced by the macrophages of organism's immune system. Therefore, the main object is to introduce into the macromolecular system an immune trap to avoid neutralization of the active substances being introduced.

### SUMMARY OF THE EMBODIMENTS

A method for making a macromolecular system for delivery of active substances with an immune trap to avoid neutralization of the substances being administered is disclosed. The method includes the steps of solubilizing the selected active substance in a solubilizer, making a suspension in the form of a nano-dispersed composition of the active substance, and introducing phosphotidylcholine into said composition and mixing with water or an aqueous solution in pre-determined ratios.

In some instances, the solubilizer can be polyethylene glycol Cremophor RH 40 or analogs thereof having a molecular weight of 40 mM. The solubilizer can be polyethylene glycol Cremophor EL or analogs thereof having a molecular weight of 35 mM. It can be polyethylene glycol Solutol HS 15 or analogs thereof having a molecular weight of 15 mM. The solubilizer can be a mixture of polyethylene glycols having different molecular weights ranging from 15 mM to 40 mM.

According to some aspects of the present disclosure, the solubilizer can be a mixture of Solutol HS 15 or analogs thereof having a molecular weight of 15 mM, Cremophor EL or analogs thereof having a molecular weight of 35 mM, and Cremophor RH 40 or analogs thereof having a molecular weight of 40 mM. The mixture of cremophors can be made at a weight ratio of Cremophor RH-40 to Cremophor EL of 2.0-2.5:0.5-1.0, and the mixture of cremophors with Solutol HS 15 is taken at a weight ratio of 3.0:1.0.

In some instances, the solubilizer can be a mixture of polyethylene glycols and poloxamers, wherein the polyethylene glycols having molecular weights ranging from 15 mM to 40 mM are taken in a mixture or each separately. The solubilizer can be a mixture of Solutol HS 15 or analogs thereof having a molecular weight of 15 mM, Cremophor EL or analogs thereof having a molecular weight of 35 mM, Cremophor RH 40 or analogs thereof having a molecular weight of 40 mM.

Other aspects, embodiments and features of the method will become apparent from the following detailed description when considered in conjunction with the accompanying figures. The accompanying figures are for schematic purposes and are not intended to be drawn to scale. In the figures, each identical or substantially similar component that is illustrated in various figures is represented by a single numeral or notation. For purposes of clarity, not every component is labeled in every figure. Nor is every component of each embodiment of the device and method shown where illustration is not necessary to allow those of ordinary skill in the art to understand the device and method.

### BRIEF DESCRIPTION OF THE DRAWINGS

The preceding summary, as well as the following detailed description of the disclosed device and method, will be better understood when read in conjunction with the attached drawings. It should be understood, however, that neither the device nor the method is limited to the precise arrangements and instrumentalities shown.

FIG. 1 is a schematic diagram illustrating a method of making an aqueous solution of the product in accordance with embodiments of the present disclosure.

FIG. 2 is a van der Waals model of micellar form of beta-carotene produced in accordance with the method of the present invention.

### DETAILED DESCRIPTION OF SPECIFIC EMBODIMENTS

The aforementioned problems are solved due to the fact that in the method for making a macromolecular system comprising at least three substances that are different in solubility in water, wherein the less soluble substance is active and solubilizes in the macromolecule of the substance, which is a solubilizer that includes the solubilization of the active agent in a solubilizer, and the third substance is phosphotidylcholine, which is embedded in the received associant with the subsequent introduction, in the resulting suspension, of water or an aqueous solution, the active substance or several active substances are pre-selected, thereafter, by solubilization of the selected active substance or several active substances in the solubilizer, a suspension in the form of nanodispersed composition of said active substance or several active substances is obtained, thereafter, water or an aqueous solution is introduced into the resulting nanoparticulate composition to produce a transparent and stable aqueous solution of the drug, the weight ratio of the active substance or several active substances to the solubilizer being taken in a weight ratio of 1.0:1.0-60.0, and the weight ratio of the phosphatidylcholine to the received associate being taken in the ratio of 1.0:1.0-50.0.

Furthermore, the aqueous solution may comprise preservatives of the parabens family and/or sorbic acid or analogs thereof.

Furthermore, the aqueous solution may comprise water-soluble C-group vitamins at a weight ratio to water of 1.0:20.0-1000.0.

Furthermore, as a solubilizer, can be used polyethylene glycol PEG-40 glyceryl tri-hydroxystearate (synonyms: macrogolglycerol hydrostearate, polyethylene glycol 40 glyceryl tri-hydroxystearate, polyoxyl 40 hydrogenated castor oil), tradename: Cremophor RH-40, or analogs thereof. It can be polyethylene glycol PEG glyceryl tri-ricinoleate (synonyms: macrogol glyceral ricinoleate, polyethylene glycol 35 glyceryl tri-ricinoleate, polyoxyl-35 castor oil), tradename: Cremophor EL, or analogs thereof.

In some instances, it can be polyethylene glycol PEG hydroxystearate (synonyms: macrogol hydroxystearate, PEG-15 hydroxystearate, polyethylene glycol 660 hydroxystearate), tradename: Solutol HS-15, or analogs thereof.

According to other aspects of the present invention, it can be a mixture of polyethylene glycols, in particular Cremophor RH-40 or analogs thereof, Cremophor EL or analogs thereof, Solutol HS-15 or analogs thereof, wherein the mixture of cremophors is selected at a weight ratio of Cremophor RH-40 to Cremophor EL to be 2.0-2.5: 0.5-1.0, and a mixture of cremophors with Solutol HS-15 is taken at a weight ratio of 3.0:1.0,

In some instances, it can be a mixture of polyethylene glycols and poloxamers, wherein each of the polyethylene glycols is taken separately or in a mixture, in particular Cremophor RH-40 or analogs thereof, Cremophor EL or analogs thereof, Solutol HS-15 or analogs thereof, wherein the mixture of cremophors is selected at a weight ratio of Cremophor RH-40 to Cremophor EL to be 2.0-2.5: 0.5-1.0, and a mixture of cremophors with Solutol HS 15 is taken at a weight ratio of 3.0:1.0, and, as poloxamers, used are: Poloxamer-188 containing 81% polyethylene glycol and 19% polypropylene glycol, tradename: Lutron F 68 or analogs thereof, or (and) Poloxamer-407 containing of 73% polyethylene glycol and 27% polypropylene glycol, tradename: Lutrol F 127 or analogs thereof, or (and) macrogol or polyethylene glycol containing 100% polyethylene glycol, tradename: Lutrol E 400 or analogs thereof, wherein one of said poloxamers is added to the polyethylene glycol separately or to a mixture of polyethylene glycols at a weight ratio of 0.1-1.0:1.0-4.0. Some brand names, synonyms and chemical names of the commercially-available solubilizers are summarized in Table 1 for clarity.

TABLE 1
Brand names of solubilizers.

| Brand Name | Synonyms | Chemical Name |
|---|---|---|
| Kolliphor RH40 | Cremophor RH40 | Macrogolglycerol |
| | | hydroxystearate or |
| | | Polyoxyl 40 Hydrogenated |
| | | Cator Oil |
| Kolliphor EL | Cremophor EL | Castor oil, ethoxylated |
| Kolliphor HS15 | Solutol HS15 | 12-Hydroxystearic acid- |
| | | polyethylene glycol |
| | | copolymer |
| Lutrol F68 | Pluronic F68 | Polyoxyethylene- |
| | | polyoxypropylene block |
| | | copolymer |
| Lutrol F127 | Pluronic F127 | Polyoxyethylene- |
| | | polyoxypropylene |
| | | Block Copolymer |
| Lutrol E400 | Kollisolv PEG400 | Polyethylene glycol 400 |
| | | Macrogol 400 |

The claimed method can utililze the following exemplary components.

Solutol HS-15 or analogs thereof, as a solubilizer, water-soluble, manufactured by BASF AKZIENGESELLSCHAFT, Germany (see Volker Buhler “Vademecum for Vitamin Formulations”, Stuttgart, Wiss.Ver1.-Ges., Birkenwaldstr., 44, D-70191, 2001, pp. 88-90, technical documentation is available at www.pharma-solutions.basf.com).

Cremophor EL or analogs thereof, as a solubilizer, water-soluble, manufactured by BASF AKZIENGESELLSCHAFT, Germany (see Volker Buhler “Vademecum for Vitamin Formulations”, Stuttgart, Wiss.Ver1.-Ges., Birkenwaldstr., 44, D-70191, 2001, pp. 88-90, technical documentation is available at www.pharma-solutions.basf.com).

Cremophor RH-40 or analogs thereof, as a solubilizer, water-soluble, manufactured by BASF AKZIENGESELLSCHAFT, Germany (see Volker Buhler “Vademecum for Vitamin Formulations”, Stuttgart, Wiss.Ver1.-Ges., Birkenwaldstr., 44, D-70191, 2001, pp. 88-90, technical documentation is available at www.pharma-solutions.basf.com).

Vitamin C, water-soluble (see Volker Buhler “Vademecum for Vitamin Formulations”, Stuttgart, Wiss.Verl.-Ges., Birkenwaldstr., 44, D-70191, 2001, pp. 14-19, technical documentation is available at www.vitamini.ru).

The preservative is selected from the products of the parabens family, for example, para-oxybenzone acid ethyl alcohol (see “Sanitary rules and regulations”, Alimentary raw materials and foodstuffs, Moscow, 2002, p. 141, E 214; Order No. 222 dated July 23, 1996. Ministry of health of Ukraine, registered with the Ministry of Justice of Ukraine on Dec. 16, 19966 ref no. 715/1740 “About approval of sanitary rules and regulations in relation to the use of food additives”, E 214).

The preservative is sorbic acid (see “Sanitary rules and regulations”, Alimentary raw materials and foodstuffs, Moscow, 2002, p. 141, E 200; Order No. 200 dated Jul. 23, 1996. Ministry of Health of Ukraine, registered with the Ministry of Justice of Ukraine on Dec. 16, 1996 ref no. 715/1740 “About approval of sanitary rules and regulations in relation to the use of food additives”, E 214).

Lipoid S75 (phosphotidylcholine), not soluble in water, soluble in oil, manufactured by Lipoid GmbH Ludwigshafen (Germany) (see technical documentation at www.lipoid.com).

Lipoid S100 (phosphotidylcholine), not soluble in water, soluble in oil, manufactured by Lipoid GmbH Ludwigshafen (Germany) (see technical documentation at www.lipoid.com).

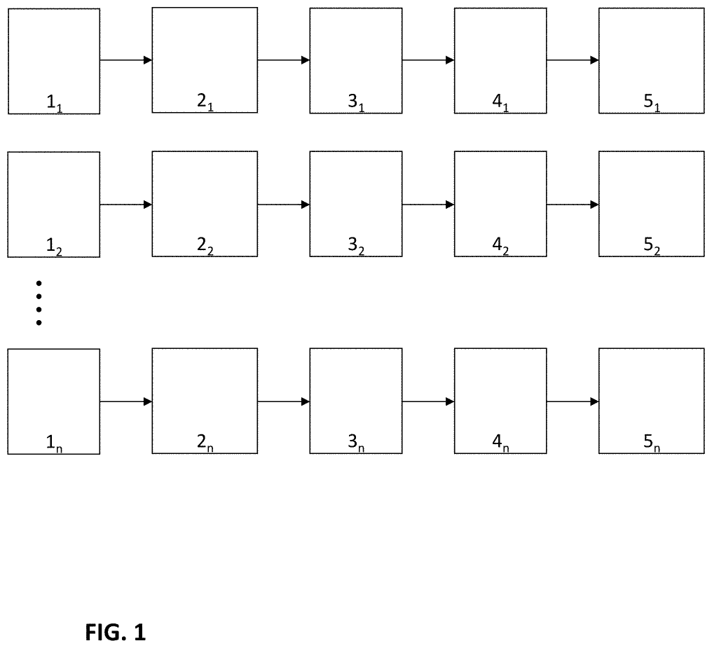

FIG. 1 illustrates a process of making an aqueous solution of the product in accordance with the present disclosure.

A water-soluble product comprising the active substance according to the subject invention is made from the selected active substance by solubilizing the selected active substance in a pre-heated to 60° C., introducing, into said composition, phosphatidylcholine, and thereafter cooling the resulting composition to 30° C., followed by introducing, into the resulting nano-dispersed composition, water or an aqueous solution comprising C-group vitamins and/or preservatives.

1. Preliminarily selecting the desired active substance (position 1₁, 1₂, 1ₙ in FIG. 1).

2. Solubilizing the selected active substance in the solubilizer (position 2₁, 2₂, 2ₙ in FIG. 1).

3. Making a suspension in the form of a nano-dispersed composition of said active substance (positions 3₁, 3₂, 3ₙ in FIG. 1).

4. Introducing, into the resulting nano-dispersed composition, water or an aqueous solution with a preservative and/or with water-soluble vitamins C and/or B, and/or a nano-dispersed composition of fat-soluble vitamins A, D2, D3, E, K1, beta-carotene (provitamin A) (positions 4₁, 4₂, 4ₙ in FIG. 1).

5. Making, as a result of dissolution, an aqueous solution of the selected active substance (positions 5₁, 5₂, 5ₙ in FIG. 1).

Making the aqueous solution of the product of the selected active substance by way of making and dissoluting in water (aqueous solution) the nano-dispersed composition of the selected active substance is described in examples.

The solubilizer, can be polyethylene glycol or a mixture of glycols, or a mixture of polyethylene glycol and poloxamer or a mixture of glycols and poloxamer, in particular Cremophor RH-40 or analogs thereof, Cremophor EL or analogs thereof, Solutol HS-15 or analogs thereof, wherein the mixture of Cremophors is selected at a weight ratio of Cremophor RH-40 to Cremophor EL to be 2.0-2.5: 0.5-1.0, and the mixture of Cremophors with Solutlon HS 15 is taken at a weight ratio of 3.0:1.0. Lutrol F68, Lutrol F127, Lutrol E400 or analogs thereof are used as poloxamers. One of the above poloxamers is added to each polyethylene glycol separately, or to a mixture of polyethylene glycols at a weight ratio of 0.1-1.0:1.0-4.0 (see Example 1, Table 2).

Therein, phosphatidylcholines Lipoid S75, Lipoid S100 or analogs thereof are used, wherein the weight ratio of phosphatidylcholines to the solubilizer is selected to be 1.0:1.0-60.0.

Furthermore, aqueous solutions comprise preservatives and/or C-group water-soluble vitamins with the weight ratios to water of 1:20.0-1000.0, and products of the parabens family and/or sorbic acid are used as a preservative (see Example 2, Table 3).

The use of the subject method for making the aqueous solutions of active substances is supported by the examples below, but is not limited thereto.

Example 1 shows the weight ratios of solubilizers and phosphotidylcholines.

Example 2 shows the ratios of the components of aqueous solutions.

Examples 3-4 describe methods for making the end product in the form of aqueous solutions.

The nano-dispersed compositions produced in accordance with the methods of the present invention are characterized by the particle size in the range from 0.01 to 300 nm, preferably in the range from 0.01 to 100 nm.

Example 1. The ratios of solubilizers and phosphatidylcholines in % wt. are shown in Table 2.

TABLE 2
Ratios of solubilizers and phosphatidylcholines, % wt.

| | Ex- | Ex- | Ex- | Ex- | Ex- | Ex- |
|---|---|---|---|---|---|---|
| | ample | ample | ample | ample | ample | ample |
| Name | 1.1 | 1.2 | 1.3 | 1.4 | 1.5 | 1.6 |
| Cremophor RH-40 | 1.0 | | | 2.0 | 2.25 | 2.5 |
| Cremophor EL | | 2.0 | | 1.0 | 0.75 | 0.5 |
| Solutol HS-15 | | | 3.0 | 1.0 | 1.0 | 1.0 |
| Lutrol F68 | | | 0.25 | | | 1.0 |
| Lutrol F127 | | 0.5 | | | | |
| Lutrol E400 | 1.0 | | | 1.0 | | |
| Lipoid S75 | 0.2 | | 3.0 | | 0.1 | |
| Lipoid S100 | | 0.1 | | 0.05 | | 4.0 |

Example 2. The ratios of aqueous solutions in % wt. are shown in Table 3.

TABLE 3
Ratios of water solutions in % wt.

| | Example | Example | Example | Example | Example | Example |
|---|---|---|---|---|---|---|
| Name | 2.1 | 2.2 | 2.3 | 2.4 | 2.5 | 2.6 |
| Paraben | 0.0005 | — | 0.0008 | 0.0008 | 0.0008 | — |
| Sorbic acid | 0.0005 | 0.0002 | 0.0002 | 0.0002 | — | 0.0005 |
| Ascorbic acid | 0.08 | 0.08 | 0.08 | — | 0.05 | 0.05 |
| Water | 1.0 | 1.0 | 1.0 | 1.0 | 1.0 | 1.0 |

Example 3. Preparation of an aqueous solution of interferon 2-alpha at 150,000 IU/ml, vitamin C at 8 mg/ml.

Interferon 2-alpha (5 million IU/mg) is selected as the active substance.

To make a solubilizer, Cremophor RH-40 and Cremophor EL are mixed at a ratio of 2.0:1.0. Thereafter, Solutol HS-15 is introduced into the resulting mixture of cremophors at a ratio of 3.0:1.0. The resulting mixture is heated to 60° C., a 10 g quantity of Lipoid S75 is added, then cooled to 30° C. and mixed with Lutrol E400 at a ratio of 1.0:1.0.

The molar ratio of the interferon 2-alpha substance to the resulting solubilizer 1.0:1.0 corresponds to a weight ratio of 1.0:20.0.

A 0.75 g quantity of the interferon 2-alpha substance is solubilized in 15 g of the solubilizer (until completely dissolved) while vigorously stirring. Therein, a nano-dispersed composition of interferon is made, which is gradually supplemented with 985 ml of an aqueous solution, in which 0.2 g of sorbic acid and 8 g of vitamin C are pre-diluted, a solution of interferon is obtained at 30° C. while stirring vigorously, in which the concentration of interferon equals to 150,000 IU/ml, that of vitamin C equals to 8 mg/ml, that of phosphatidylcholine equals to 10 mg/ml.

Example 4. Preparation of an aqueous solution of porcine insulin at 20 IU/ml, vitamin C at 8 mg/ml. The substance of porcine insulin at 24 IU/mg is selected as the active substance. To make a solubilizer, Cremophor RH-40 and Cremophor EL are mixed at a ratio of 2.0:1.0. Thereafter, Solutol HS-15 is introduced into the resulting mixture of cremophors at a ratio of 3.0:1.0. The resulting mixture is heated to 60° C., a 10 g quantity of Lipoid S100 is added, then cooled to 30° C. and mixed with Lutrol F127 at a ratio of 1.0:1.0.

The molar ratio of 1.0:1.0 of the porcine insulin substance to the resulting solubilizer corresponds to a weight ratio of 1.0: 20.0. A 0.84 g quantity of the porcine insulin substance is solubilized in 17 g of the solubilizer (until completely dissolved) while vigorously stirring. Therein, a nano-dispersed composition of porcine insulin is made, which is gradually supplemented with 982 ml of the aqueous solution, in which 8 g of vitamin C, 0.2 g of sorbic acid are pre-diluted, a solution of insulin is made at 30° C. while stirring vigorously, in which the concentration of insulin equals to 20 million IU/ml, that of vitamin C equals to 8 mg/ml, that of phosphatidylcholine equals to 10 mg/ml.

Example 5. Preparation of an aqueous solution of beta-carotene 20 IU/ml, vitamin E at 8 mg/ml. The substance of 100% crystalline beta-carotene and 100% oil solution of alpha-tocopherol acetate (vitamin E) are selected as the active substances. To make a solubilizer, Cremophor RH-40 is heated to 65° C. and mixed at a ratio 4.0:1.0 with 2 grams of beta-carotne and 0.8 grams alpha-tocopherol acetate (vitamin E), vigorously stirring for 20 minutes, and then the resulted mixture is added to 100 ml of water heated to 65° C. and stirred for 20 minutes.

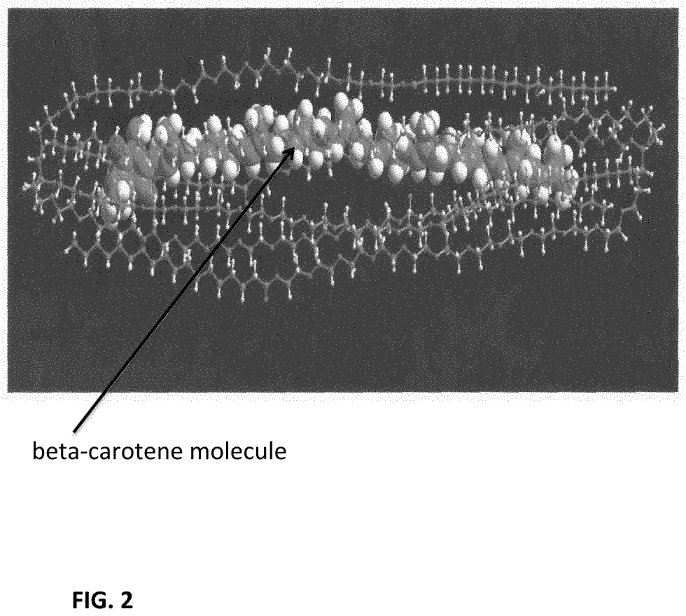

The van der Waals model of micellar form of beta-carotene produced in accordance with the method of the present invention is shown in FIG. 2.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

Although the invention is described herein with reference to specific embodiments, various modifications and changes can be made without departing from the scope of the present invention as set forth in the claims below. Accordingly, the specification and figures are to be regarded in an illustrative rather than a restrictive sense, and all such modifications are intended to be included within the scope of the present invention. Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements.

The foregoing detailed description is merely exemplary in nature and is not intended to limit the invention or application and uses of the invention. Furthermore, there is no intention to be bound by any expressed or implied theory presented in the preceding technical field, background, brief summary, or the following detailed description.

What is claimed is:

1. A method for making a macromolecular system for delivery of active substances with an immune trap to avoid neutralization of the substances being administered, the method comprising:
solubilizing the selected active substance in a solubilizer;
making a suspension in the form of a nano-dispersed composition of said active substance; and
introducing phosphotidylcholine into said composition and mixing with water or an aqueous solution in pre-determined ratios.

2. The method as claimed in claim 1, wherein the solubilizer is polyethylene glycol Cremophor RH 40 or analogs thereof having a molecular weight of 40 mM.

3. The method as claimed in claim 1, wherein the solubilizer is polyethylene glycol Cremophor EL or analogs thereof having a molecular weight of 35 mM.

4. The method as claimed in claim 1, wherein the solubilizer is polyethylene glycol Solutol HS 15 or analogs thereof having a molecular weight of 15 mM.

5. The method as claimed in claim 1, wherein the solubilizer is a mixture of polyethylene glycols having different molecular weights ranging from 15 mM to 40 mM.

6. The method as claimed in claim 5, wherein the mixture is selected from the group consisting of Solutol HS 15 or analogs thereof having a molecular weight of 15 mM, Cremophor EL or analogs thereof having a molecular weight of 35 mM, and Cremophor RH 40 or analogs thereof having a molecular weight of 40 mM;

7. The method as claimed in claim 6, wherein the mixture of cremophors is made at a weight ratio of Cremophor RH-40 to Cremophor EL of 2.0-2.5:0.5-1.0, and the mixture of cremophors with Solutol HS 15 is taken at a weight ratio of 3.0:1.0.

8. The method as claimed in claim 1, wherein the solubilizer is a mixture of polyethylene glycols and poloxamers, wherein the polyethylene glycols having molecular weights ranging from 15 mM to 40 mM are taken in a mixture or each separately.

9. The method as claimed in claim 8, wherein the solubilizer is a mixture of Solutol HS 15 or analogs thereof having a molecular weight of 15 mM, Cremophor EL or analogs thereof having a molecular weight of 35 mM, Cremophor RH 40 or analogs thereof having a molecular weight of 40 mM.

10. The method as claimed in Clam 9, wherein the mixture of cremophors is made at a weight ratio of Cremophor RH-40 to Cremophor EL of 2.0-2.5:0.5-1.0, and the mixture of cremophors with Solutol HS 15 is taken at a weight ratio of 3.0:1.0.

11. The method as claimed in claim 8, wherein the poloxamers are selected from the group consisting of Lutrol F68, Lutrol F127, Lutrol E 400 or analogs thereof; and wherein one of said poloxamers is added to polyethylene glycol separately or to a mixture of polyethylene glycols at a weight ratio of 0.1-1.0:1.0-4.0.

12. The method as claimed in claim 1, wherein the phosphatidylcholine is Lipoid S75 or analogs thereof having a molecular weight of 0.7 mM.

13. The method as claimed in claim 1, wherein the phosphatidylcholine is Lipoid S100 or analogs thereof having a molecular weight of 0.7 mM.

14. The method as claimed in claim 1, wherein the water or aqueous solutions containing at least one of preservatives, antioxidants and water-soluble C-group vitamins in pre-determined ratios.

15. The method as claimed in claim 1, wherein the preservative is selected from the group consisting of products of the parabens family, sorbic acid and analogs thereof.

16. The method as claimed in claim 1, wherein the particle size of the nano-dispersed composition is in the range from 0.01 nm to 100 nm.

17. The method as claimed in claim 1, wherein the active substance is 100% crystalline beta-carotene.

18. The method as claimed in claim 17, wherein the active substance further comprising 100% oil solution of alpha-tocopherol acetate (vitamin E).

19. The method as claimed in claim 1, wherein the active substance is porcine insulin.

20. The method as claimed in claim 1, wherein the active substance is interferon 2-alpha.
